(19) United States
(12) Patent Application Publication
(10) Pub. No.: US 2013/0202540 A1
(43) Pub. Date: Aug. 8, 2013

(54) METHOD FOR REPAIR OF ACUTE AND CHRONIC INJURY, SUCH AS BURNED AND PHOTODAMAGED SKIN

(71) Applicant: Madalene C.Y. Heng, Northridge, CA (US)

(72) Inventor: Madalene C.Y. Heng, Northridge, CA (US)

(21) Appl. No.: 13/368,429

(22) Filed: Feb. 8, 2012

(51) Int. Cl. A61K 8/35; A61Q 19/00; A61K 8/41; A61K 8/97; A61K 8/34; A61K 8/49

(52) U.S. Cl. 424/59

(57) ABSTRACT

A chemical compound and a method for its use as a topical medication that can reverse the effects of sun-damage, reverse the signs of aging, demonstrate anti-scarring properties and allow for the treatment of acne while still permitting the patient the ability to simultaneously expose the affected area to the sun without damage that includes a combination of curcumin, alcohol, carbomer, diazolidinylurea, additional penetration enhancer—such as alliin, additional agent capable of blocking both UVA and UVB such as pearl powder, anti-inflammatory agent such as aloe vera, water, glycerin and preservative as needed, and an acidifying agent to product the correct pH. Allinin is used as an s-allyl side chain to increase the penetration of curcumin into the cells. Pearl powder is used to block the effects harmful UVA and UVB.

### BACKGROUND OF THE INVENTION

1. Field of the Invention

The present invention relates generally to the field of dermatology, and particularly toward a method for the repair of acute injury, including burns scalds and sunburn, and chronic injury, such as photodamaged skin, i.e., actinic keratoses, solar lentigenes and prelentigo maligna, solar elastoses, actinic poikiloderma, hyperpigmentation and wrinkling, using special formulations of topical curcumin to achieve the following: (a) the induction of the rapid repair of burns, scalds and sunburn without scarring, i.e., perfect regeneration, by the inhibition of inflammation by blocking curcumin-targeted phosphorylase kinase/TGFiβ signaling pathways; (b) the induction of the repair of photodamaged skin, i.e., actinic keratoses, solar lentigenes, premalignant lentigo maligna, solar elastoses, actinic poikiloderma (thinning of the skin with telangiectasia), hyperpigmentation and wrinkling, by a slow process of curcumin-induced apoptosis of damaged precancerous cells, and replacing the damaged cells with healthy cells.

2. Description of the Prior Art

Garlic is a spice, with a varied number of properties, including anticarcinogenic properties, cardioprotective properties, anti-viral, anti-microbial and antifungal properties. The active ingredient in garlic is alliin (S-allyl cysteine sulfoxide), which is metabolized to allicin (N-acetyl-S allyl-cysteine) by an enzyme released when its membranes are crushed. Other metabolites include allyl mercaptan, diallyl disulfide, diallyl sulfide, diallyl sulfoxide, diallyl sulfone and methyl sulfide.

Garlic has been thought to bring about its anti-carcinogenic effect through a number of mechanisms, such as the scavenging of free radicals by increasing free radical quenchers, like glutathione and catalases, and the prevention of chromosomal damage and increasing DNA repair mechanisms, as well as by inducing apoptosis (cell death) of cancer cells.

Cardiac protective properties have been linked to vasoactivity of garlic, which has been linked to the mediation by hydrogen sulfide. In addition, its hypocholesterterolemic effect has been linked to the suppression of HMG-reductase expression and inhibition of cholesterol synthesis.

Allicin has been found to have antiviral properties as well as antifungal properties. Moreover, allicin has been shown to enhance the oxidative damage of Amphotericin B against Candida albicans. In addition, the antifungal properties of allicin have also been linked to increased intracellular ergosterol trafficking.

The ability of allicin to permeate phospholipids membrane has been observed. More recently, garlic allyl derivatives have been reported to interact with membrane lipids to modify the membrane fluidity. However, the notion of using the lipid permeability properties of the S-allyl molecules in garlic and DMSO to serve as carrier molecules to transport non-lipid soluble molecules across cell membranes, has not hitherto been reported.

Injury to the skin triggers a cascade of injury-induced inflammatory and repair processes. Acute injury such as burns and scalds frequently result in undesirable consequences such as blistering and bullae formation, swelling and erythema, inflicting considerable pain to the sufferer, and resulting in loss of function. Sunlight may also cause acute sunburns, resulting in pain, erythema and blistering. Burns from ultraviolet light injury are due mainly to the ultraviolet B spectrum (290-320 nm wavelength).

Burns and scalds are caused by heat injury to the cellular proteins, with resultant coagulative necrosis of the cells, and damage to cellular proteins both within the cytoplasm and nucleus. Damage to the DNA in the nucleus sets up the well-known DNA damage response (DDR) in an attempt to repair the DNA. However, the accompanying inhibition of cell proliferation i.e. cell cycle arrest, associated with the DNA damage response impairs the ability of the cells to regenerate new cells, thus slowing down the repair process Furthermore, the inflammatory process in burns and scalds induce cytokines, such as TGFβ1, which results in hypertrophic scar formation, which is commonly observed with second and third degree burns and scalds. The formation of hypertrophic scars involves the conversion of fibroblasts to myofibroblasts, which is induced by secretion of excessive TGFβ.

The damaging wavelengths in sunlight are usually attributed to wavelengths in the ultraviolet range i.e. ultraviolet B or UVB (290-320 nm wavelength), and ultraviolet A or UVA (320-400 nm wavelength). The ultraviolet C component of solar radiation (200-280 nm) is filtered off by clouds, humidity, dust particles etc., and is thought not to be a significant factor on Earth. Although UVB wavelengths are more prone to cause sunburn, current evidence suggests that UVA radiation, which makes up 95% of the solar ultraviolet light reaching the earth, may be the more damaging of the two with regard to carcinogenesis and photo-aging. Additionally, other wavelengths, such as infra red rays that produce heat, may also contribute to the injury observed in acute sunburns and chronic dermal injury.

Radiation in the UVB range is less penetrating than wavelengths in the UVA range. Thus, UVB radiation is only capable of penetrating down to the mid-epidermis. Consequently, UVB is only implicated in the induction of squamous cell carcinomas since these cells are situated more superficially than basal cells and melanocytes. On the other hand, UVA penetrates deep into the dermis, and are responsible for the formation of basal cell carcinomas and malignant melanomas, as well as for dermal changes of photo-aging, such as wrinkling and loss of elasticity associated with solar elastosis. Clothing and sunscreens which block UVB, do not block UVA, and these agents should not be relied upon for prevention of photodamaged skin.

Point mutations and mutagenic CPDs (cyclobutane pyrimidine dimers) have been associated with both UVB and UVA exposure. However, the CPDs produced by UVB, tend to be pyrimidine dimers containing thymine-cytosine, cytosine-thymine, cytosine-cytosine, which are easily removed and produce limited injury to the DNA. On the other hand, the CPDs produced by UVA tend to be predominantly thymine-thymine pyrimidine dimers, which are difficult to remove, and tend to produce damage to large segments of the DNA. The damage induced by the large double stranded DNA breaks, which induce the DNA response pathways, are difficult to repair, and frequently result in errors of replication which cause cells to transform into their malignant counterparts. In addition, it has been observed that the bystander effect, which results in tissue damage outside the areas exposed, was only observed with UVA radiation but not with UVB.

Since sunscreens have been shown to be somewhat ineffective in the prevention of photocarcinogenesis, attention has been focused on certain botanicals, in particular curcumin for the treatment of photo-aging skin and prevention of photocarcinogenesis. Curcumin (diferuloylmethane) is an active ingredient in the spice, turmeric. The effectiveness of curcumin administered orally is hindered by its poor bioavailability due to the fact that the unconjugated curcumin molecule, which is hydrophobic, is poorly absorbed when taken orally, with poor bioavailability in blood and tissues. However, the curcumin product in a topical gel base, has been found to be effective in skin disease.

Topical curcumin is an anti-inflammatory agent with the ability to block the inflammatory response following acute and chronic skin injury through blocking NF-kB signaling pathways through inhibition of phosphorylase kinase. In acute injury such as burns, there is abrogation of the inflammatory response with inhibition of NF-kB/TGFβ signaling pathways with the use of curcumin gel, resulting in rapid healing of the burn injury without residual scarring. In chronic injury from repeated ultraviolet light damage, the photodamaged skin may be repaired by curcumin-induced apoptosis of DNA-damaged precancerous cells, allowing for a more gradual replacement of the damaged cells by new healthy, undamaged cells. The instant invention provides a method for the rapid repair of burn injury without scarring following curcumin gel therapy, as well as slow improvement, of photodamaged skin with actinic keratoses, solar elastoses, actinic poikiloderma (thinning of the skin with telangiectasia), dysplastic nevi and solar lentigenes with the same treatment.

It is the object of the instant invention to utilize improved methods of topical absorption of curcumin.

### SUMMARY OF THE INVENTION

By using a mixture of alliin and allicin in Allium sativum (garlic) extract as a carrier molecule, we can enhance the penetration of non-lipid soluble medicaments, such as 1% xylocaine to rapidly penetrate cell membranes, including skin membranes (stratum corneum), so that numbness can be produced by merely painting a mixture of the Allium sativum extract combined with 1% xylocaine on the skin.

Furthermore, the instant invention discloses using Allium sativum extract (or alliin/allicin or analogs) to enhance curcumin through the skin, such as for the treatment of deep injury such as keloidal scars and photoaging skin.

Furthermore, the instant invention discloses using Allium sativum extract (or alliin/allicin or analogs) to enhance non-lipid soluble medicaments through the skin for topical use. These include antiviral agents, such as acyclovir, valacyclovir and famciclovir, antifungal agents such as terbenafin, griseofulvin (anti-fungal), clotrimazole or ketoconazole, and topical antibiotics, such as clindamycin, neomycin, polysporin, bacitracin etc.

Furthermore, the instant invention discloses using Allium sativum extract (or alliin/allicin and analogs) to enhance non-lipid soluble medicaments, including curcumin, to penetrate the skin to treat superficial arthritis.

Furthermore, the instant invention discloses using purified alliin/allicin or analogs to enhance curcumin packaged in microspheres through target organ cell membranes, such as liver, brain, or joints, with potential use in cancer, Alzheimer's disease and arthritis.

Furthermore, the instant invention discloses using alliin/allicin or analogs in an inhaler to deliver non-lipid soluble medicaments to the sinuses or lungs.

Furthermore, the instant invention discloses using Allium sativum extract (alliin/allicin or analogs) to deliver non-lipid soluble medicaments to mucous membranes.

The preferred embodiment of the instant invention provides for a method for the early treatment of acute skin injury, such as burns, scald, sunburn and the like that also treats chronic skin injury such as photo-damaged skin (actinic keratoses, solar lentigenes, solar elastoses, dysplastic nevi, prelentigo maligna, pigmentary changes, telangiectasia (actinic poikiloderma) and wrinkling) in order to induce rapid healing of damaged tissue without scarring comprising the steps of: the application to said damaged tissue of a topical preparation of curcumin, such as curcumin gel, containing the following: (a) curcumin (0.0001-30%) (b) alcohol (0.001-20%) (c) carbomer (d) diazolidinylurea (e) additional penetration enhancer—such as alliin (f) additional agent capable of blocking both UVA and UVB such as pearl powder (g) anti-inflammatory agent such as aloe vera (h) water, glycerin and preservative as needed (i) acidifying agent to product the correct

### BRIEF DESCRIPTION OF THE DRAWINGS

FIG. 1 is a table demonstrating the signaling targets blocked by Curcumin in NF-kB dependent signaling in acute injury.

FIG. 2 is a table demonstrating signaling targets in chronic solar injury inhibited by curcumin.

FIG. 3 is a table demonstrating the details of sites of phosphorylase involved in activation of IkB Kinase. This kinase contains three sub-units, α, β and γ (also called NEMO). The γ subunit (NEMO) contains a zinc finger and ubiquitin ligase site, which is involved in the DNA response pathway. Activation of IkB kinase is blocked by curcumin.

FIG. 4A is a photograph showing a two year old with at least second degree burns on both hands after falling into a camp-fire. He was seen 4 days later treated with silvadene cream.

FIG. 4B is a photograph showing improvement when seen 24 hours later after hourly application of curcumin gel. Patient was also put on oral prednisone for 2 weeks.

FIG. 4C is a photograph showing rapid healing with curcumin gel treatment (frequent applications) when seen 2 weeks later. Oral corticosteroid therapy had been stopped by his patents by this time.

FIG. 4D is a photograph showing the same patient with burns treated with frequent applications of curcumin gel, showing complete healing without erythema or scarring when seen two months later.

FIG. 5A is a photograph showing photo-damaged skin with multiple confluent solar lentigenes and actinic keratoses on the dorsum both hands and forearms before curcumin gel.

FIG. 5B is a photograph showing improvement in the photodamaged skin over the dorsum of both hands and extensor aspects of both forearms after 15 months of treatment.

FIG. 6 is a photograph showing shows severely photo-damaged skin before (left panels), and 12 months following application of extra-strength curcumin gel (right panels). Note improvement in texture, solar lentigenes, actinic keratoses of large sheets of skin following treatment with curcumin gel (right panels).

FIG. 7 is a photograph showing photo-damaged skin with severe wrinkling and solar elastosis before curcumin gel treatment (left panel). Note improvement in wrinkling and solar elastosis after 16 months with curcumin gel and sunscreen (right panel).

FIG. 8 is a photograph showing severely photo-damaged skin with marked thinning and telangiectasia (actinic poikiloderma) before the use of curcumin gel (top panel). Improvement is observed with curcumin gel applied twice daily after three months (middle panel), with greater improvement nine months after curcumin gel therapy bottom panel).

FIG. 9 is a photograph showing Melasma i.e. hyperpigmentation in sun-exposed distribution of cheeks and face (upper panel) induced by cytokine-induced photosensitivity associated with underlying lactose intolerance is improved (lower panel) with a lactose free diet, topical curcumin during the day, and more sunscreen on the dark areas than over the light areas.

FIG. 10 shows in the upper panel, a photograph of a dysplastic nevus showing an irregular pigmentation with the nevus. Note the erythema due to dilated blood vessels associated with an inflammatory response directed against the premalignant cells. The lower panel shows a photograph of the same nevus 6 months after treatment with curcumin gel. Note the absence of erythema indicative of removal of the premalignant cells by curcumin-induced apoptosis.

FIG. 11 shows in the upper panel, a photograph of a dysplastic nevus with irregularity in shape and pigmentation showing erythema from an inflammatory response directed against the precancerous cells prior to curcumin gel treatment. The lower panel is a photograph of the same nevus 6 months after treatment with curcumin gel. Note the absence of erythema indicative of removal of premalignant cells by curcumin-induced apoptosis.

FIG. 12 is a photograph showing shows the forearm of a patient with severely photodamaged skin with multiple solar lentigenes. The central lesion, which may represent early prelentigo rnaligna, shows irregularity in shape and pigmentation, demonstrates erythema. In the lower panel, notice the improvement in erythema, irregularity and pigmentation of the treated lesions after 6 months of curcumin gel applied twice daily. Also note general improvement in skin texture with the treatment.

### DETAILED DESCRIPTION OF A PREFERRED EMBODIMENT

Below is a discussion of the differences and similarities between the signaling pathways in acute injury such as burns, resulting mainly in scarring, and chronic solar damage, resulting in photo-damaged skin and photo-carcinogenesis, pointing out the common.

signaling targets blocked by curcumin in its ability to repair both acute and chronic injury.

### (1) Signaling Pathways Induced by Acute and Chronic Injury

### (A) NF-kB Dependent Signaling Pathways

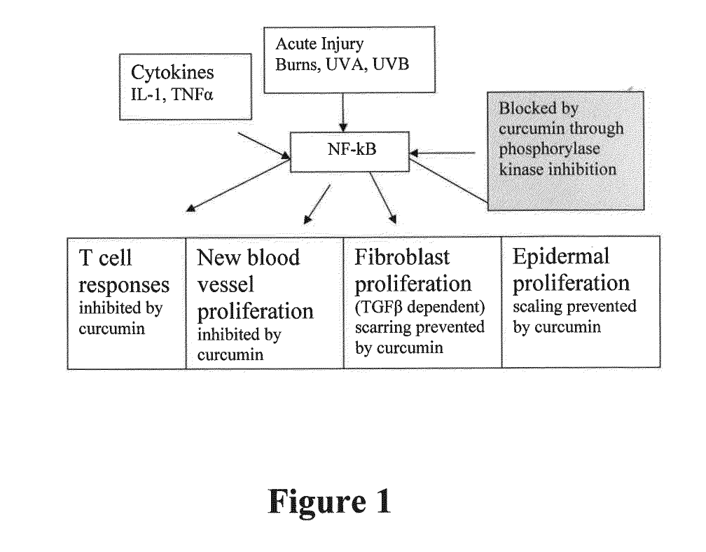

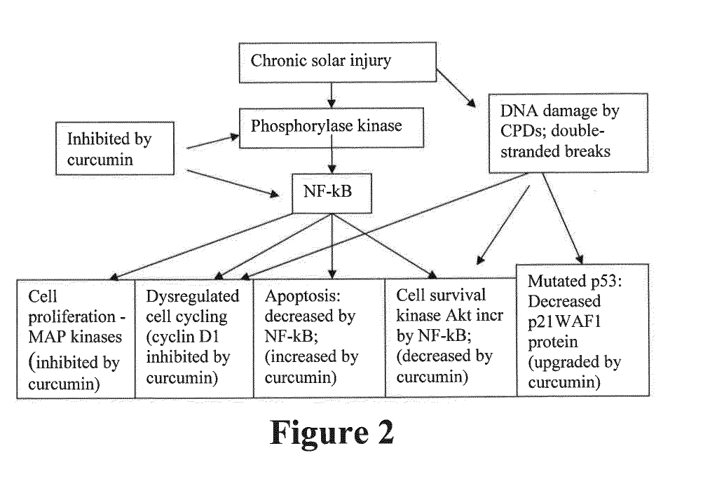

In acute burns and sunburns, the injury stimulus results in inflammatory processes, such as T cell activation. The repair processes, resulting in the production of new blood vessels, and fibroblastic proliferation, lead to dermal scarring (FIG. 1). With repeated insults to the skin with chronic solar injury, damage to the epidermis result in epidermal proliferation and scaly skin, and melanocytic proliferation leading to the formation of premalignant solar lentigenes and dysplastic nevi. Additionally, with chronic solar damage, DNA injury may result in photocarcinogenesis, with dysregulated cell cycling and malignant transformation, leading to squamous cell carcinomas, basal cell carcinomas and malignant melanomas (lentigo rnaligna, superficial spreading melanomas, and nodular melanomas). The above pathways, mediated by NF-kB dependent signaling pathways, and inhibited by curcumin are shown in FIGS. 1 and 2.

### (a) Role of NF-kB in Injury Pathways

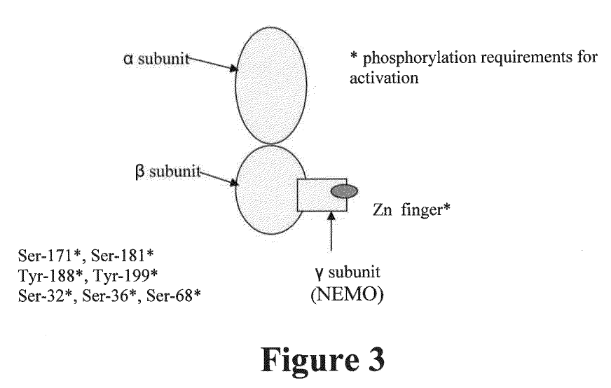

Curcumin, the active ingredient in the spice, turmeric, is an indirect but potent inhibition of NF-kB activation. Following injury, gene transcription is induced by the activation of transcription regulators. One of the major transcription regulators is nuclear factor kappa B or NF-kB. NF-kB belongs to a family of related protein dimers that bind to a common sequence on the DNA known as the kB site. In the unactivated state, NF-kB exists as a pair of dimers (p50 and p65) located within the cytoplasm. After activation by injury, these dimers translocate to the nucleus, where they bind to the DNA, and are responsible for activation of multiple genes related to cell proliferation, cell migration, neovascularization, scar tissue formation, inhibition of apoptosis, stimulation of cell survival kinase (Akt), enhancement of dysregulated cell cycling and decreased expression of the p53 suppressor gene (FIG. 3). The p53 suppressor gene encodes the p21WAF1 protein, which binds to both strands of the DNA during DNA replication, thus stabilizing the DNA and prevents dysregulated proliferation. DNA damaged by UV radiation results in decreased expression of p53, resulting in decreased production of p21WAF1 protein, with decreased ability to stabilize DNA strands during replication. This results in dysregulated proliferation and malignant transformation. Curcumin has been shown to inhibit cell cycle progression by upregulating p21WAF-1 and p53.

### (b) Activation of NF-kB and IkBa Kinase

The activation of NF-kB is triggered by injurious stimuli, including ultraviolet light radiation. In the unactivated state, NF-kB exists as a pair of dimers located within the cytoplasm. Following injury, activation of NF-kB involves phosphorylation at three serine specific sites (Ser-276, Ser-529 and Ser-536). In addition, before the NF-kB can translocate to the nucleus, the inhibitory molecule, IkBa needs to be removed by activation of the enzyme IkBa kinase. IkBa kinase consists of three subunits (α, β subunits, and γ subunit (NEMO) which contains a zinc finger, with an ubiquitin-ligase binding site). Activation of IkB kinase requires activation of sites which are both serine specific and tyrosine specific: Ser-171, Ser-181³⁵ and Tyr-188, Tyr 199, on the β subunit, as well as phosphorylation of the zinc finger on the γ subunit. In ultraviolet light-induced injury, additional sites (Ser-32,Ser-36, Ser 68) are also phosphorylated. The zinc finger of the γ subunit (NEMO)) is selectively required for NF-kB activation by ultraviolet light radiation. The removal of the inhibitory IkBa molecule through activation of its kinase, IkBa kinase, enables the activated NF-kB dimers to translocate to the nucleus, where it is responsible for activating genes such as mitogen-activated protein kinases (MAP kinases), which cause proliferation of damaged cells. Ultraviolet light injury may also activate several MAP kinase signaling pathways. Besides activating NF-kB, UV light injury may also induce the activation of transcription factors such as AP-1 (fos and jun) resulting in activation of the p38 MAPK pathway. Curcumin has also been shown to inhibit the c-jun N-terminal kinase (JNK) signaling. In addition, curcumin also blocks NF-kB and ERK signaling.

NF-kB promotes carcinogenesis in skin and tissues by increasing the cell survival kinase (Akt, a serine-threonine kinase, and other NF-kB-dependent cell survival genes involving survivin, TRAF₁ and TRAF₂, which block apoptosis of photodamaged cells. Activation of NF-kB allows DNA-damaged and potentially malignant cells to survive. By blocking Akt and cell survival proteins, curcumin induces apoptosis of the DNA damaged cells, with resultant anti-carcinogenic effect.

### (c) Role of Phosphorylase Kinase in NF-kB and MD Kinase Activation: Blocked by Curcumin

Phosphorylase kinase in a unique kinase in which the spatial arrangements of the specificity determinants can be manipulated to allow phosphorylase kinase to transfer high energy phosphate bonds from ATP to substrates of different specificities, such as serine/threonine and tyrosine. This is achieved by the presence of a hinge joint between the subunits of phosphorylase kinase, which allow changes in the substrate binding site, as well as the ability to change the shape of the substrate binding site by binding either to Mg++ or Mn++ ions. Phosphorylation of multiple serine specific sites (Ser276, Ser529 and Ser536) on the NF-kB molecule is necessary for the initial partial activation of NF-kB. Additionally, phosphorylation of multiple serine specific (Ser171, Ser 181) and tyrosine specific (Tyr 188, Tyr198) on the IkBa kinase molecule is necessary for the removal of the inhibitory molecule (IkBa), in order that the activated NF-kB may translocate to the nucleus to bind to the DNA for gene transcription. The multiple phosphorylations of differing moieties such as serine, threonine and tyrosine, may thus be achieved through the activity of phosphorylase kinase alone. In addition, the use of one enzyme ensures that the phosphorylation reactions are synchronized. Activation of NF-kB is associated with, transcription of multiple genes related to inflammation, cell proliferation, scarring, and malignant transformation. The effects of phosphorylase kinase activation are therefore responsible for much of the aftermath of injury-triggered disease. The utilization of a single enzyme (PhK) for phosphorylation of multiple serine/threonine and tyrosine-specific sites has the advantage for synchronization of phosphorylation of multiple sites of different specificities, required for the activation of NF-kB and its inhibitor protein, IkB kinase. It is possible that phosphorylase kinase may also be involved in inhibition of cyclin D1 by curcumin, leading to enhancement of its anti-carcinogenic properties.

Curcumin, the principal ingredient in the spice, turmeric, is a specific and non-competitive inhibitor of phosphorylase kinase. Its clinical use in a wide range of different skin diseases is achieved through its inhibitory effect on phosphorylase kinase. It thus appears that curcumin, through PhK inhibition, may function as an indirect inhibitor of NF-kB activation and NF-kB-dependent injury pathways kinase. This includes blockage of cell proliferation by inhibition of MAP kinases (which are made of both serine/threonine kinases (MAP kinase, kinase kinase, and MAP kinase kinase) and tyrosine kinases (MAP kinase), and induces apoptosis by blockade of Akt (a serine threonine cell survival kinase). This removes potentially malignant cells from the damaged tissue. Apoptosis of damaged cells by curcumin is necessary to allow the space for new cells to be formed. In photo-damaged skin, the formation of new cells replaces the damaged cells, thus allowing repair of the damaged tissue.

### (e) Use of Curcumin Gel in the Repair of Burns and Photodamaged Skin

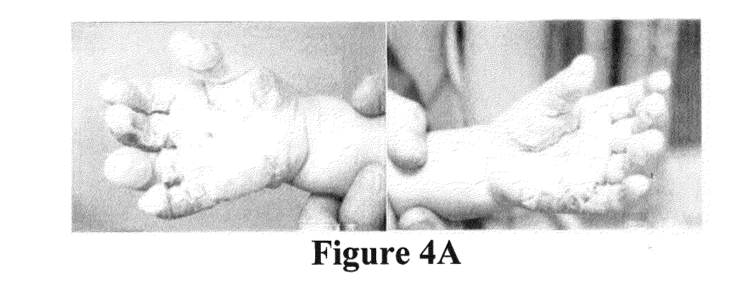

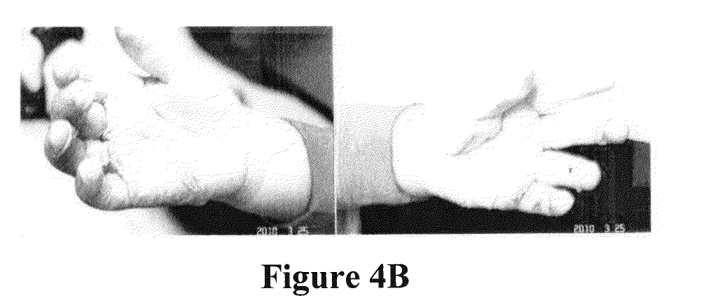

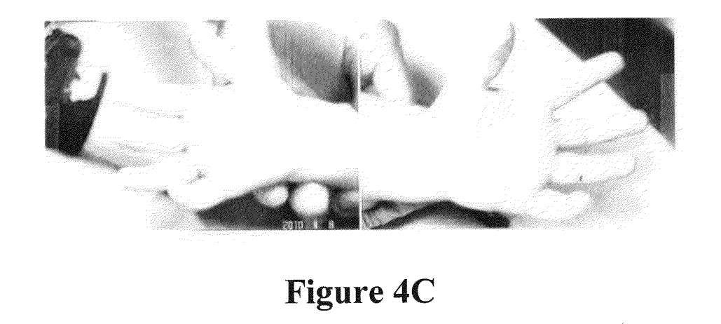

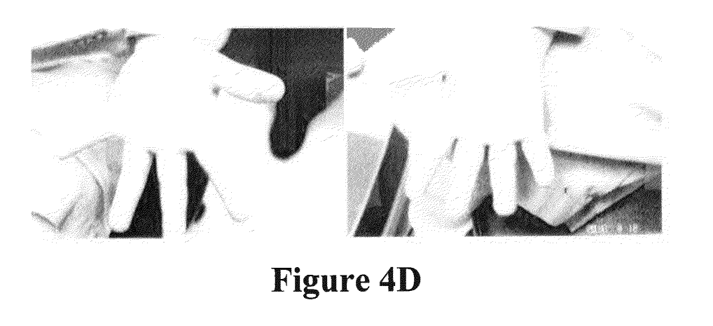

In acute burns, the removal of damaged cells by apoptosis allows room for more rapid healing by replacement of the damaged cells by new healthy cells. In addition, blockade of the NF-kB-dependent signaling prevents overgrowth of excessive scar tissue, which usually accompanies severe burns. With curcumin gel, the burns are observed to heal rapidly with no scarring and apparent perfect regeneration (FIGS. 4A-4D).

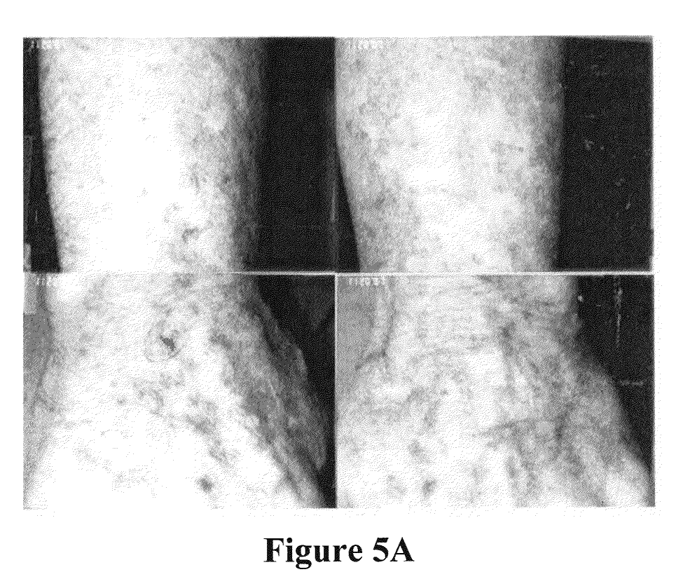

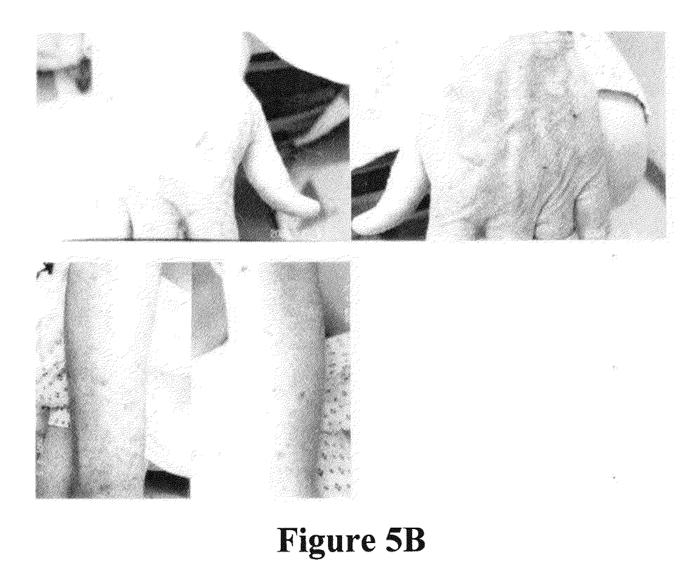

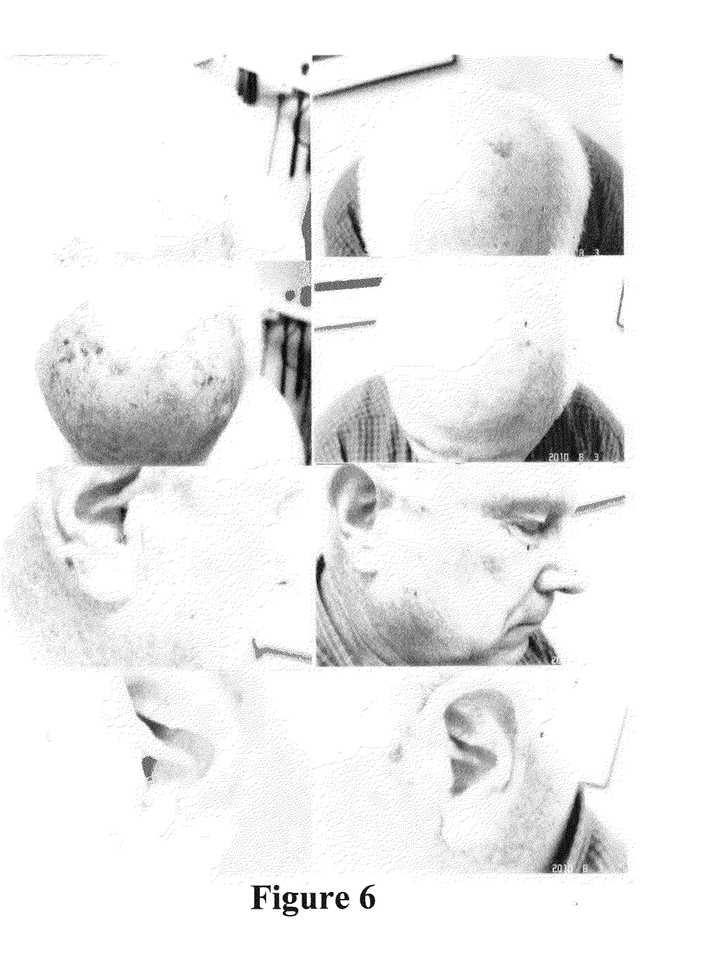

Similarly, the removal of damaged cells in chronically solar damaged skin by apoptosis prevents the DNA damaged cells from surviving to develop into precancerous and cancer cells. The replacement of the damaged cells by new healthy cells allows the skin to repair photo-damaged areas (FIGS. 5A-5B). This modality also allows for the successful repair of large areas of photo-damaged skin without surgical intervention. The obvious advantage of removal of precancerous cells produced by chronic solar damage by curcumin-induced apoptosis allows for potential treatment of precancerous lesions, including actinic keratoses and solar lentigenes, by non-surgical methods (FIGS. 5A-5B; FIG. 6).

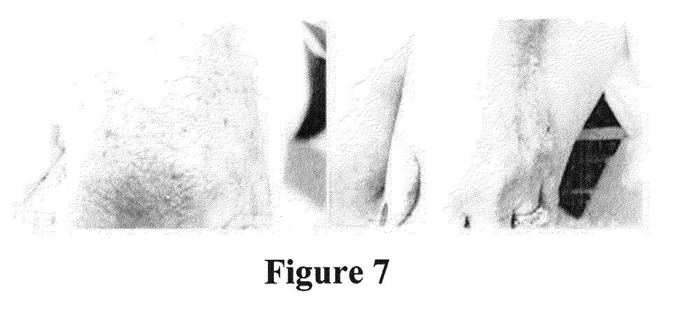

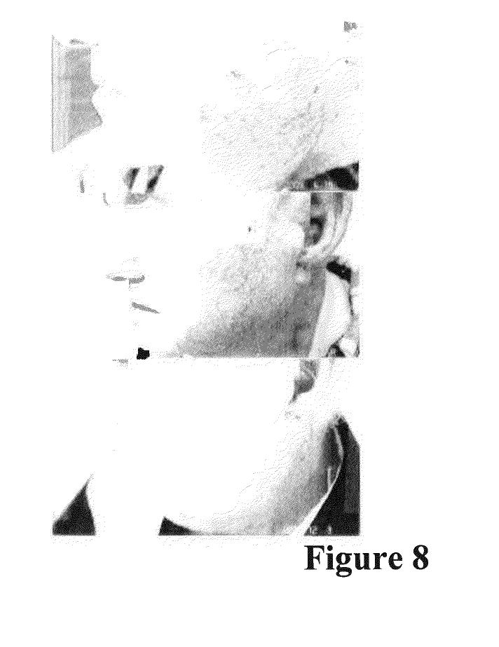

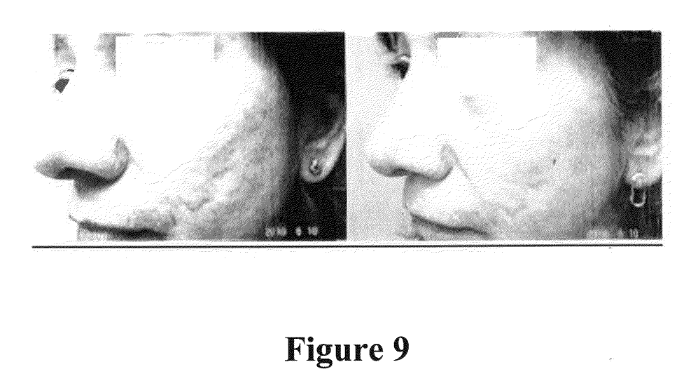

Furthermore, curcumin gel may also be useful in the repair of many of the features found in chronic solar injury (photo-damaged skin), including improvement in texture and solar elastosis (FIG. 7). It has also been observed that application of curcumin gel results in improvement in actinic poikiloderma (thinning of the skin and telangiectasia; FIG. 8). Furthermore, application of curcumin gel over the entire face, with application of sunscreen over the areas of melasma, have been observed to result in improvement of the areas of hyperpigmentation (FIG. 9).

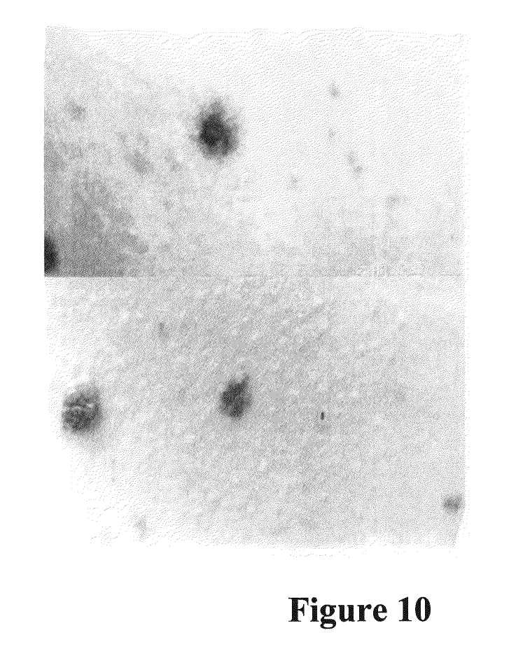

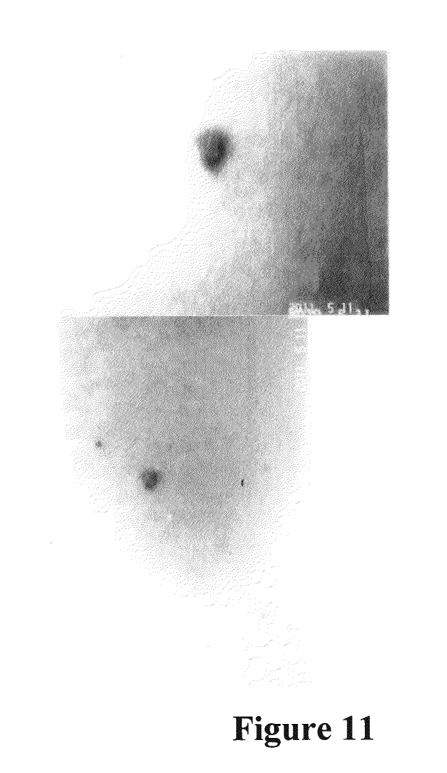

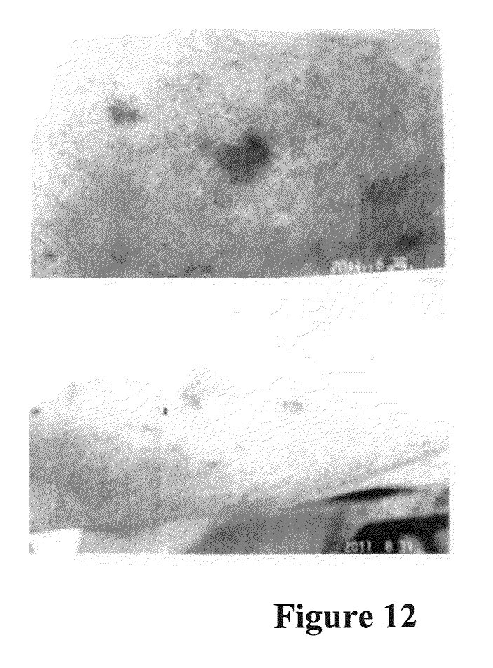

In patients with dyplastic nevi and advanced solar lentigenes, the use of a good camera capable of taking close-up shots, may be used to reveal erythema (FIGS. 10-12) due to the presence of inflammatory response directed against premalignant cells within the dysplastic nevi/advanced solar lentigenes. Resolution of the erythema within 6 months was observed after curcumin gel applied twice daily for 6 months.

Many medicaments, including xylocaine, a local anesthetic, are incapable of penetration through undamaged cell membranes such as the stratum corneum. It is, therefore, not currently feasible to deliver topical xylocaine directly to the skin without a needle to penetrate the stratum corneum.

We have discovered that by using alliin and/or allicin in Allium sativum (garlic) extract, we can enhance the penetration of topical 1% xylocaine through the stratum coneum such that numbing of the skin is produced by merely painting a mixture of Allium sativum extract and 1% xylocaine in a gel medium on the skin. Following the contact with this mixture, the skin is numbed so that a subsequent injection of 1% xylocaine delivered into the subcutaneous tissue causes very little discomfort to the patient. In the same patient, 1% xylocaine injected subcutaneously in control sites not previously treated with the Allium sativum/1% xylocaine extract produced pain and discomfort to the patient.

Since alliin (S-allyl cysteine sulfoxide) and allicin ((N-acetyl-S allyl-cysteine or diallylthiosulfinate) in Allium sativum (garlic) extract has the capability to dissolve in lipids, this may contribute to its ability to penetrate cell membranes, which are composed of a two layers of phospholipids. However, since 1% xylocaine is not lipid soluble, the local anesthetic does not have the capability of penetrating the lipid-containing cell membranes, including the stratum corneum. Using a mixture of 1% xylocaine and Allium sativum extract containing both alliin and allicin, in a gel medium for easy application to the skin with a Q tip, we are able to use the Allium sativum extract as a carrier molecule to enhance the penetration of a non-lipid soluble medicament, such as 1% xylocaine, rapidly (within 1 minute) through lipid cell membranes, including the stratum corneum (Heng MCY; unpublished data, 2010). We have found that other S-allyl containing small molecules such as dimethylsulfoxide (DMSO) can also function in the same way.

Curcumin is the active ingredient in the instant mixture. In its pure form, curcumin is not absorbed through the skin. The instant invention provides a formulation that allows for absorption, of curcumin through the skin with efficacy. Our formulation provides the curcumin, solubilized in alcohol, with the right milieu to penetrate the layers of the stratum corneum, which constitutes the skin barrier. The stratum corneum barrier is made up of 10 (forearm)-200 (palms and soles) layers of stratum corneum, and consists of alternating layers with hydrophilic properties and hydrophobic properties (desmoglein, lipid bi-layer consisting of phospholipids and a lipid core, for each of the 10-200 layers). We have found that addition of other ingredients to our formulation results in retardation of penetration of curcumin through the stratum corneum barrier, with decreased salutary effects of the clinical outcomes. We have found that the optimum milieu depends on the concentration of curcumin, concentration of alcohol, pH, concentrations of carbomer, alliin, aloe vera, diazolidinylurea, as well as the proportion of these ingredients, together with lack of interference of this milieu by additional ingredients. The optimization of this milieu is shown by salutary effects of our topical curcumin preparation in producing optimal clinical efficacy as shown in FIGS. 3-12.
1. The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

1. A method for the early treatment of acute skin injury, such as burns, scalds, sunburn and the like that also treats chronic skin injury such as photo-damaged skin (actinic keratoses, solar lentigenes, solar elastoses, dysplastic nevi, prelentigo maligna, pigmentary changes, telangiectasia (actinic poikiloderma) and wrinkling) in order to induce rapid healing of damaged tissue without scarring comprising the steps of:
the application to said damaged tissue of a topical preparation of curcumin, such as curcumin gel, containing the following:
(a) curcumin (0.0001-30% by weight of the total composition)
(b) alcohol (0.001- 20% by weight of the total composition)
(c) carbomer
(d) diazolidinylurea
(e) additional penetration enhancer—such as alliin
(f) additional agent capable of blocking both UVA and UVB such as pearl powder
(g) anti-inflammatory agent such as aloe vera
(h) water, glycerin and preservative as needed
(i) acidifying agent to produce the correct pH.
